(19) United States
(12) Patent Application Publication
(10) Pub. No.: US 2025/0205273 A1
(43) Pub. Date: Jun. 26, 2025

(54) ANTIVIRAL COMPOSITION COMPRISING CHITOSAN AND XANTHOHUMOLONE

(71) Applicant: HEALTH PLUS, BEGLES (FR)

(72) Inventors: Saadia MOATACIM, BEGLES (FR); Gilbert BAUER, BEGLES (FR)

(21) Appl. No.: 18/850,215

(22) PCT Filed: Mar. 27, 2023
(86) PCT No.: PCT/EP2023/057840

§ 371 Date: Oct. 24, 2024

(30) Foreign Application Priority Data
Mar. 31, 2022 (FR) FR2202932

(51) Int. Cl. A61K 31/722; A61K 31/12; A61P 31/14

(52) U.S. Cl. CPC A61K 31/722; A61K 31/12; A61P 31/14

(57) ABSTRACT

The present invention relates to a composition comprising at least chitosan and xanthohumolone, to a process for preparing same and to the uses thereof. More particularly, the invention relates to a composition comprising at least chitosan and xanthohumolone for use as an antiviral composition, in particular in the treatment or prevention of COVID-19.

### TECHNICAL FIELD

The invention relates to the technical field of antiviral compositions. The invention relates in particular to a composition comprising at least chitosan and xanthohumolone, to a process for preparing same and to the uses thereof.

### PRIOR ART

December 2019 marked the start of the COVID-19 pandemic, which has since spread around the world with over 350 million cases and more than 5.5 million deaths.

With the virus spreading at breakneck speed, numerous scientific teams around the world have been hard at work finding solutions to limit its spread.

According to the World Health Organization (WHO), the virus can be spread when small liquid particles are expelled through the mouth or nose when an infected person coughs, sneezes, talks or breathes deeply. These particles range in size from large “respiratory droplets” to smaller “aerosols”.

The COVID-19 virus, like many airborne viruses, is transmitted mainly by respiratory droplets. These droplets loaded with viral particles could infect a potential subject either through direct contact with a mucous membrane (direct transmission) or through contact with an infected surface via the nasal, buccal or conjunctival mucosa (indirect transmission). They can be projected several meters away, but do not persist in the air.

Once inhaled, to infect its host, the COVID-19 virus attaches itself to a protein present on the surface of cells, notably lung cells: the ACE2 receptor. This attachment then enables it to penetrate the cell and infect its host.

One of the strategies envisaged is to prevent the virus from entering the cell by interfering with the mechanisms required for the virus to bind to its receptor.

In addition to isolation, ventilation and barrier measures, the most widespread solution is to wear a mask during close contact to prevent the transmission of droplets and aerosols.

There are three main types of masks on the market to combat the transmission of airborne pathogens.

FFP (Filtering Facepiece) masks are classified according to their ability to filter aerosols, and comply with the NF EN 149 standard.
FFP1 (80% aerosol filtration)
FFP2 (94% aerosol filtration)
FFP3 (99% aerosol filtration)

This type of mask protects the wearer from inhaling airborne particles, not to mention larger droplets that could contain infectious agents.

There are also surgical masks that comply with the NF EN 14683 standard. By preventing the projection of droplets emitted by the mask wearer, this type of mask limits contamination of the external environment and other people. There are also several types of surgical masks, classified according to their robustness.

And finally, there are the so-called “consumer” masks, which are textile masks with guaranteed filtration, mostly washable and reusable. They are easily recognizable. They are reserved for use outside the healthcare system.

“Consumer” masks have filtration properties of over 90% of 3-micron particles for category 1, or over 70% of the same particles for category 2.

All masks on the market are effective in limiting the spread of airborne viruses, including the SARS-Cov-2 responsible for COVID-19, but also all have the same disadvantages, namely:
very limited comfort when breathing, especially during prolonged use,
a significant amount of waste due to their short lifespan,
masks have no specific action against viruses; they are merely a physical barrier.

With the emergence of pandemics such as COVID-19, there is a great need to find new solutions to limit the spread of viruses, especially airborne viruses such as COVID-19.

One of the solutions proposed by the prior art is the combination of an antiviral vaccine with a means of preventing the spread of viruses.

However, there are currently no effective solutions for limiting the spread of airborne viruses, while at the same time having a protective antiviral action.

There is therefore a need for a means of protection against the spread and infection of viruses, particularly airborne viruses such as COVID-19.

### SUMMARY OF THE INVENTION

To meet this need, the invention proposes a new composition comprising a mixture of at least chitosan and xanthohumolone.

Chitosan is a natural biopolymer derived from chitin, notably from crustaceans.

Xanthohumolone is a flavonoid derived from hops, in particular the Humulus Lupulus species.

Chitosan and xanthohumolone are molecules that have long been used in a number of fields, including food, cosmetics and pharmaceuticals. Their safety profile and innocuousness are already known in the prior art.

Surprisingly, the inventors found that combining chitosan and xanthohumolone produced a composition with a synergistic antiviral effect, particularly against airborne viruses such as COVID-19.

Such a composition is particularly useful in the context of the invention and overcomes the drawbacks of the prior art, being biocompatible and exhibiting significant antiviral activity. Moreover, the residues resulting from the degradation of the composition according to the invention are neither toxic nor genotoxic, and do not induce inflammation, particularly of the skin or mucous membranes.

The composition according to the invention has a xantohumolone/chitosan ratio of between 0.2 and 0.7, preferentially between 0.4 and 0.6, even more preferentially 0.5.

According to one embodiment, the invention relates to a composition comprising a mixture of chitosan and xanthohumolone, together with a biocompatible solvent such as acetic acid.

The composition according to the invention is suitable for use in human or animal health, preferentially for use as an antiviral or antibacterial composition.

According to another aspect, the composition according to the invention is also suitable for the treatment of medical devices.

Advantageously, the composition according to the invention can be used to disinfect and protect medical devices to prevent the spread of viruses and/or bacteria.

The invention further relates to a kit comprising a vaccine and a composition comprising the combination of chitosan and xanthohumolone.

According to a final aspect, the invention further relates to a process for preparing the composition according to the invention, comprising in particular a step of dissolving chitosan, mixing, heating and adding a solution of xanthohumolone.

Further features and advantages will become apparent from the detailed description of the invention and the examples, which are illustrative only and by no means limit the scope of the invention.

### DETAILED DESCRIPTION OF THE INVENTION

### Definition

For the purposes of this invention, “COVID-19” refers to the SARS-COV-2 coronavirus and all its variants.

“Chitosan” within the meaning of the invention means a copolymer composed of 2-acetamido-2-deoxy-β-D-glucose and 2-amino-2-deoxy-β-D-glucose in variable proportions. Depending on the degree of deacetylation, the molecule is called chitin or chitosan. Chitin, in its natural state, is itself deacetylated to 5 or 15%. It is generally accepted that an amine content of over 7% distinguishes chitosan from chitin.

“Microfilm” within the meaning of the invention means a physical protective barrier of a biopolymer layer that acts mechanically.

“Biocompatible solvent” within the meaning of the invention means a solvent that is compatible with biological tissues. The solvent is therefore harmless to the organism.

“Cytopathic effect” within the meaning of the invention means alterations.

### Composition According to the Invention

The object of the present invention is therefore a composition comprising a mixture of at least chitosan and xanthohumolone.

Advantageously, the composition according to the invention forms a microfilm with significant antiviral activity, particularly against COVID 19.

Advantageously, the combination of chitosan and xanthohumolone has a synergistic antiviral effect.

A further advantage is that the composition according to the invention is natural, biodegradable and resorbable.

Preferably, the composition according to the invention also comprises at least one biocompatible solvent. The biocompatible solvent of the composition according to the invention can be chosen from acetic acid, glycolic acid, lactic acid, glutamic acid, glycerol and mixtures thereof. Preferentially, the biocompatible solvent is acetic acid.

Advantageously, the biocompatible solvent according to the invention enables the combination of chitosan and xanthohumolone to be solubilized, facilitating administration.

Preferably, the composition according to the invention comprises chitosan with a viscosity of between 35 and 150 cP, preferentially a viscosity of between 80 and 100 cP, in particular between 50 and 96 cP, even more preferentially between 85 and 95 cP.

For the purposes of this invention, viscosity is preferentially measured using a Brookfield apparatus at room temperature. This is a dynamic viscosity measurement method well known to the person skilled in the art.

Advantageously, the viscosity of chitosan according to the invention makes it possible to obtain a homogeneous composition.

Preferentially, the composition according to the invention comprises chitosan with a molecular weight of between 150 and 500 KDa, preferentially between 200 and 300 KDa, even more preferentially between 225 and 275 KDa.

Advantageously, according to the invention, chitosan with a molecular weight of between 150 and 500 KDa has significant antibacterial and antiviral activity.

Preferably, the composition according to the invention comprises chitosan with a degree of deacetylation of between 70 and 90%, preferentially between 85 and 90%.

According to a particular embodiment of the invention, the composition according to the invention, with or without biocompatible solvent, also comprises at least one additive.

The additive according to the invention is preferentially chosen from perfume, essential oils and anionic substances.

When the composition according to the invention comprises an additive such as an anionic substance, the additive can be chosen from Na(2)SO(4) and Na(2)HPO(4).

According to a particularly preferred embodiment, the composition according to the invention comprises:
Between 0.0001 and 0.025% xanthohumolone by weight
Between 0.1 and 10% chitosan by weight
Optionally between 0.1 and 5% additives by weight

Preferably, the composition according to the invention has a xantohumolone/chitosan ratio of between 0.2 and 0.7, preferentially between 0.4 and 0.6, even more preferentially 0.5.

Advantageously, such a ratio results in enhanced antiviral and antibacterial activity.

The composition according to the invention may be in the form of a gel, solution, cream or spray, preferentially in the form of a spray, particularly a nasal spray.

Advantageously, when the composition is in nasal spray form, it offers high antiviral efficacy in a single application. In addition, the microfilm formed by the composition according to the invention is very easily resorbable in the nasal mucosa. Once applied, the composition coats the nasal mucosa, preventing viruses or bacteria from attaching to the cells by means of physical and chemical protection via the production of an antiviral and antibacterial microfilm.

According to another aspect, the invention relates to a medical device comprising the composition according to the invention. Preferentially, the medical device according to the invention can be a mask such as a surgical mask, an FFP mask or a cloth mask.

Process for preparing the composition according to the invention

According to another aspect, the composition according to the invention comprises at least the implementation of the following steps:
a) Dissolving chitosan
b) Mixing, with stirring, the dissolved chitosan from step a)
c) Adjusting the pH of the mixture from step b)
d) Heating the mixture from step c)
e) Adding xanthohumolone solution to the mixture from step d)

Preferably, the chitosan is dissolved in a biocompatible solvent at a concentration of between 0.2 and 2% at a ratio of 0.1:1, 1:1, or 2:1.

The biocompatible solvent of step a) can be chosen from acetic acid, glycolic acid, lactic acid, glutamic acid, glycerol and mixtures thereof.

Preferentially the biocompatible solvent in step a) is acetic acid.

Preferably, the mixing in step b) is carried out in a paddle mixer at a rotational speed of between 40 and 150 rpm (rpm=revolutions per minute), preferentially between 50 and 100 rpm.

The mixing in step b) is preferentially carried out at room temperature.

According to a preferred embodiment, the mixing in step b) is carried out for 4 to 12h, preferentially for 6 to 8h.

The pH of step c) is adjusted by adding a base to the mixture of step b) until a pH of between 5 and 7, preferentially 5.5 to 6.5, is obtained.

Heating in step d) is carried out at a temperature of between 3° and 100° C., preferentially between 8° and 90° C.

Preferentially, the heating in step d) is carried out in a water bath.

In a preferred embodiment, the heating in step d) is carried out for a period of 15 minutes to 1 hour, preferentially 30 minutes.

Preferentially, the heating in step d) is carried out with gentle stirring.

According to a particularly preferred embodiment, the xanthohumolone solution from step e) is added at a concentration of between 10 and 100 μg/ml, preferentially between 40 and 70 μg/ml.

### Kit:

According to another aspect, the invention relates to a kit comprising the composition according to the invention and a vaccine.

The various kit components can be used simultaneously or sequentially.

Preferably, the vaccine is an antiviral vaccine, preferentially an anti-COVID-19 vaccine.

According to a particular embodiment, the kit according to the invention comprises a vaccine selected from: Tozinaméran (Pfizer-BioNTech), mRNA-1273 (Moderna), Gam-COVID-Vac (Spoutnik V), AZD1222 (Oxford-AstraZeneca), Ad26.COV2.S (Janssen/Johnson & Johnson), Ad5-nCOV/Convidecia (CanSino Biologics), BBIBP-CorV (Sinopharm), WIBP (Sinopharm), CoronaVac (Sinovac Biotech), BBV152/Covaxin (Bharat Biotech), CoviVac (Chumakov), ZF2001/RBD-Dimer (Anhui Zhifei Longcom), EpiVacCorona (Vector).

In a particularly preferred embodiment, the kit comprises a nasally administered vaccine, such as FLUENZ TETRA (AstraZeneca).

### Uses

According to a final aspect, the invention relates to the composition according to the invention for use as a medicament, preferentially as an antiviral composition or antibacterial composition.

The composition according to the invention can be used as an antiviral or antibacterial drug.

Thus, another object of the invention is a composition for use in the prevention and/or treatment of viral and bacterial diseases, preferentially for preventing and/or combating COVID-19, herpes simplex 1 and 2, cytomegalovirus, hepatitis A, papillomavirus, human immunodeficiency virus as well as sexually transmitted diseases.

Examples include the prevention and/or treatment of influenza, for which the influenza virus is responsible, as well as the prevention and/or treatment of severe acute respiratory syndrome (SARS), Middle East respiratory syndrome (MERS), COVID-19 disease, for which coronaviruses are responsible, and the prevention and/or treatment of angina, for which adenoviruses or bacteria such as staphylococcus and streptococcus are responsible.

Preferably, the composition according to the invention is an antiviral drug for use in the treatment or prevention of COVID-19.

Advantageously, the composition forms a microfilm on the skin or mucous membranes, preventing the virus from attaching to cells and providing antiviral activity against it.

According to another embodiment, the invention is aimed at the use of a composition as described in the present application to treat medical devices, preferentially masks such as surgical masks, FFP masks and cloth masks.

Advantageously, treating masks with the composition according to the invention enables masks or visors to be upgraded by increasing their time of use and providing them with antiviral activity, particularly against COVID-19.

According to a particular embodiment, the composition according to the invention can be used as a food supplement.

Thus, the composition according to the invention can be integrated into another composition, in particular into a nutritional composition provided in a form chosen from tablets, capsules, gelatin capsules, powders, solutions, microcapsules, suspensions, emulsions, food supplements, drinks and food for humans or animals.

### EXAMPLES

Example composition according to the invention

### Example 1

The composition 1 has the following characteristics:
Chitosan (260 KDa and 80 cP)=1% by weight of the composition
Xanthohumolone=0.025% by weight of the composition
Essential oil=0.1% by weight of the composition.

The pH of the composition is adjusted to be between 5.1 and 6.

### Example 2

The composition 2 has the following characteristics:
Chitosan (260 KDa and 80 cP)=1% by weight of the composition
Xanthohumolone=0.025%
Acetic acid=0.5
Essential oil=2%

The pH of the composition is adjusted to be between 5.1 and 6.

### Example 3

The composition 1 has the following characteristics:
Chitosan (150 KDa and 50 cP)=0.5% by weight of the composition
Xanthohumolone=0.005%
Anionic substance (Na(2)SO(4) and Na(2)HPO(4))=2 to 6%.

The pH of the composition is adjusted to be between 5.5 and 6.2.

Example process for preparing a composition according to the invention:

### Example 4

The preparation process comprises the implementation of the following steps:
Dissolving chitosan in a 2% solution of acetic acid at a ratio of 1:1.
Mixing at room temperature with stirring at 100 rpm for 7 hours
Adjusting the pH to 5.5 to 6.2 by adding NaOH
Heating in a water bath at 80° C. for 4 hours
Adding 20 to 100 μg/ml xanthohumolone solution

### Example 5

The preparation process comprises the implementation of the following steps:
Dissolving chitosan in a 1% solution of acetic acid at a ratio of 2:1.
Mixing at room temperature with stirring at 80 rpm for 6 hours
Adjusting the pH to 5.5 to 6.2 by adding NaOH
Heating in a water bath at 45° C. for 3 hours
Adding 20 μg/ml xanthohumolone solution

### Example 6

### Testing Antiviral Efficacy on a Coronavirus 229E Strain

The composition according to the invention has been formulated as a gel and tested on a coronavirus 229E strain according to NF 14476+A2 dated July 2019.

### Test Method

### A) Viruses

Name: HCOV 229 E
Product type: Animal viruses
Classification: Coronaviridae, Coronavirus
Strain designation: 229E

### B) Cells

Name: VERO
Product type: Animal cell
Organism: Cercopithecus aethiops
Morphology: epithelial
Tissue: Kidney
Physiological condition: normal

### C) Experimental Conditions

Test concentrations: 80% (V/V) of a composition according to the invention
comprising 1% chitosan and a Xanthohumolone content of 50 μg/mL
Contact time: 60 minutes±10 seconds
Interfering substance: 0.3 g/L BSA
Saline phosphate buffer: PBS
Product thinner: Water for injection
Culture medium: EMEM
Molecular sieve-filter: Séphadex LH20
Appearance of product dilutions: Liquid

Stability and appearance of the mixture during testing: Homogeneous preparation for the duration of the test

### Titration of Viral Suspension:

Titration is carried out in microplates using the suspended cell method in accordance with NF EN 14476+A2. The cytopathic effect is determined after 4 days of culture.

The estimated number of infectious units is determined by the SPAERMAN-KARBER method, well known to the person skilled in the art. The SPAERMAN-KÄRBER method involves testing according to a geometric progression, i.e. with a constant pace between successive dilutions, and inoculating a constant volume of each dilution.

This method is used to determine the Ig TCID50, that is the dilution of the virus solution causing cytopathic effects on 50% of cells in culture.

Efficiency is checked by stopping the reaction in ice for 30 minutes before virus titration is carried out.

Direct cytotoxicity test: This test reveals the concentration of the composition required to induce toxicity in the VERO cell line.

### Indirect Cytotoxicity Test: Molecular Sieving

Stopping the activity of the composition according to the invention is achieved by the molecular sieving technique using a sieve filter, that is Sephadex LH 20. The neutralization of the composition according to the invention is assessed by running it through Sephadex after an intermediate dilution. The neutralization of the product is validated by running the 1/10 dilution through Sephadex. The neutralization of Formaldehyde is validated by running the 1/20 dilution through Sephadex.

### Cell Sensitivity to Viruses:

Comparative titrations are carried out on cells treated or not with the composition according to the invention at a subcytotoxic concentration in order to check that it does not modify the behavior of the receptor cells towards the virus.

### Results:

1) Test virus suspension titre: Ig TCID50:7.1
2) Test viral control: Ig TCID50=4.88
3) Direct cytotoxicity: non-cytotoxic concentration starting at 8*10-1% (weight/volume)
4) Indirect cytotoxicity: non-cytotoxic concentration starting at 4*10-1% (weight/volume)

### Cell Sensitivity to Viruses:

Condition a: VERO cells in the presence of the composition according to the invention: Ig TCID50=5.75
Condition b: VERO cells in the absence of the composition according to the invention: Ig TCID50=5.63
Condition b-Condition a=0.12<1 lg

The sensitivity is therefore valid.

Checking the effectiveness of disinfectant shutdown at T=0
Condition a: VERO cells in the presence of the composition according to the invention: Ig TCID50=6.25
Condition b: VERO cells in the absence of the composition according to the invention: Ig TCID50=5.88
Difference <0.5, therefore valid control.

### Test Result:

TABLE 1
Coronavirus 229 E - 60 minutes ± 10 seconds

| | Composition according to the | | | |
|---|---|---|---|---|
| | invention | | | |
| | Degree of | % of | Control | |
| | cytopathic | positive | Degree of | % of |
| −log | effect | wells | cythopathic | positive |
| dilution | 80%(V/V) | | effect | wells |
| 1 | 11100000 | 37.5% | 11111111 | 100% |
| 2 | 11000000 | 25% | 11111111 | 100% |
| 3 | 00000000 | 0% | 11111111 | 100% |
| 4 | 00000000 | 0% | 11111110 | 87.5% |
| 5 | 00000000 | 0% | 11110000 | 50% |
| 6 | 00000000 | 0% | 000000000 | 0% |
| 7 | 00000000 | 0% | 000000000 | 0% |
| 8 | 00000000 | 0% | 000000000 | 0% |
| 9 | 00000000 | 0% | 000000000 | 0% |
| 10 | 00000000 | 0% | 000000000 | 0% |
| Lg | | 1.13 | | 4.88 |
| TCID50 | | | | |

* 1 to 4: Presence of virus, with cytopathic effect observed in 8 cell wells 0: Virus-free, with no cytopathic effect

### Conclusion:

According to the protocol of the NF EN 14476+A2 standard, the composition according to the invention induces a reduction in the viral load of coronavirus 229 E of 3.75 lg or 99.98% after 60 minutes' exposure.

### Example 7

Determining the virucidal activity of chitosan and xanthohulomone products according to the protocol of the NF EN 14476+A2 standard (July 2019) coronavirus 229 E.

### Sample Identification:

Composition 1:0.1% chitosan
Composition 2: 1% chitosan
Composition 3: 1% chitosan+2 mg/mL hops
Composition 4: 1% chitosan+50 μg hops

Appearance: liquid gel with deposits for compositions 3 and 4.
Composition 5:5 μg/mL Xanthohumolone
Composition 6:50 μg/mL Xanthohumolone
Composition 7:250 μg/mL Xanthohumolone

Product appearance: Solid to be reconstituted in 60% ethanol solution at 5 mg/ml

### Test Method

### A) Viruses

Name: HCOV 229 E
Product type: Animal viruses
Classification: Coronaviridae, Coronavirus
Strain designation: 229E

### B) Cells

Name: VERO
Product type: Animal cell
Organism: Cercopithecus aethiops
Morphology: epithelial
Tissue: Kidney
Physiological condition: normal

### B) Experimental Conditions

Test concentrations: 80% (V/V) of a composition according to the invention comprising 1% chitosan and a Xanthohumolone content of 50 μg/mL
Contact time: 60 minutes±10 seconds
Saline phosphate buffer: PBS
Product thinner: Water for injection
Culture medium: EMEM
Molecular sieve-filter: Séphadex LH20
Appearance of product dilutions: Liquid
Stability and appearance of the mixture during testing: Homogeneous preparation for the duration of the test

### Titration of Viral Suspension:

Titration is carried out in microplates using the cell suspension method. The cytopathic effect is determined after 4 days of culture.

The estimated number of infectious units is determined by the SPAERMAN-KARBER method, well known to the person skilled in the art. The SPAERMAN-KÄRBER method involves testing according to a geometric progression, i.e. with a constant pace between successive dilutions, and inoculating a constant volume of each dilution.

This method is used to determine the Ig TCID50, that is the dilution of the virus solution causing cytopathic effects on 50% of cells in culture.

Efficiency is checked by stopping the reaction in ice for 30 minutes before virus titration is carried out.

### Direct Cytotoxicity Test:

This test reveals the concentration of the composition required to induce toxicity in the VERO cell line.

### Indirect Cytotoxicity Test: Molecular Sieving

Stopping the activity of the composition according to the invention is achieved by the molecular sieving technique using a sieve filter, that is Sephadex LH 20. The neutralization of the composition according to the invention is assessed by passing it through Sephadex after an intermediate dilution. Product neutralization is validated by running the 1:10 dilution through Sephadex. Formaldehyde neutralization is validated by running the 1:20 dilution through Sephadex.

### Cell Sensitivity to Viruses:

Comparative titrations are carried out on cells treated or not with the composition according to the invention at a subcytotoxic concentration in order to check that it does not modify the behavior of the receptor cells towards the virus.

### Results of Compositions 1 to 4:

1) Direct cytotoxicity: non-cytotoxic concentration starting at 1*10-3% (volume/volume)
2) Indirect cytotoxicity: non-cytotoxic concentration starting at 1*10-2% (volume/volume)

### Sensitivity of Cells to Viruses of Compositions 1 to 4:

Condition a: VERO cells in the presence of the composition according to the invention: Ig TCID50=7.38
Condition b: VERO cells in the absence of the composition according to the invention: Ig TCID50=7.13
Condition b−Condition a=0.25<1 lg

The sensitivity is therefore valid.
Checking the effectiveness of disinfectant shutdown at T=0
Condition a: VERO cells in the presence of the composition according to the invention: Ig TCID50=6.25
Condition b: VERO cells in the absence of the composition according to the invention: Ig TCID50=6.88
Difference <0.5 lg, therefore valid control.

### Test Viral Control:

Before Sephadex: Ig TCID50=6
After Sephadex: Ig TCID50=5.75
   Difference=0.25<0.5 lg so the control is valid

### Test Result:

TABLE 2
Coronavirus 229 E - 60 minutes ± 10 seconds

| | In the presence of compositions 1 or 2 | | | | Control | |
|---|---|---|---|---|---|---|
| | Degree of cytopathic | | % of positive | | Degree of | % of |
| | effect | | wells | | cythopathic | positive |
| −log dilution | 2 | 1 | 2 | 1 | effect | wells |
| 1 | 11111111 | 11111111 | 100% | 100% | 11111111 | 100% |
| 2 | 11111111 | 11111111 | 100% | 100% | 11111111 | 100% |
| 3 | 11111111 | 11111111 | 100% | 100% | 11111111 | 100% |
| 4 | 11111111 | 11111111 | 100% | 100% | 11111111 | 100% |
| 5 | 11100000 | 11111000 | 37.5% | 62.5% | 11111110 | 87.5% |
| 6 | 00000000 | 00000000 | 0% | 0% | 11100000 | 37; 5% |
| 7 | 00000000 | 00000000 | 0% | 0% | 000000000 | 0% |
| 8 | 00000000 | 00000000 | 0% | 0% | 000000000 | 0% |
| Lg TCID50 | | | 4.88 | 5.12 | | 5.75 |

* 1 to 4: Presence of virus, with cytopathic effect observed in 8 cell wells 0: Virus-free, with no cytopathic effect

TABLE 3
Coronavirus 229 E - 60 minutes ± 10 seconds

| | In the presence of compositions 3 or 4 | | | | Control | |
|---|---|---|---|---|---|---|
| | Degree of cytopathic | | % of positive | | Degree of | % of |
| | effect | | wells | | cythopathic | positive |
| −log dilution | 3 | 4 | 3 | 4 | effect | wells |
| 1 | 11111111 | 11111111 | 100% | 100% | 11111111 | 100% |
| 2 | 11111111 | 11111111 | 100% | 100% | 11111111 | 100% |
| 3 | 11111111 | 11111111 | 100% | 100% | 11111111 | 100% |
| 4 | 11111000 | 11111000 | 62.5% | 62.5% | 11111111 | 100% |
| 5 | 10000000 | 11100000 | 12.5% | 37.5% | 11111110 | 87.5% |
| 6 | 00000000 | 00000000 | 0% | 0% | 11100000 | 37; 5% |
| 7 | 00000000 | 00000000 | 0% | 0% | 000000000 | 0% |
| 8 | 00000000 | 00000000 | 0% | 0% | 000000000 | 0% |
| Lg TCID50 | | | 4.25 | 4.50 | | 5.75 |

* 1 to 4: Presence of virus, with cytopathic effect observed in 8 cell wells 0: Virus-free, with no cytopathic effect

### Results of Compositions 5 to 7:

1) Direct cytotoxicity: non-cytotoxic concentration starting at 2.5*10-1 μg/ml (weight/volume)
2) Indirect cytotoxicity: non-cytotoxic concentration starting at 2.5 μg/ml (weight/volume)

Sensitivity of cells to viruses of compositions 5 to 7:
Condition a: VERO cells in the presence of the composition according to the invention: Ig TCID50=7.13
Condition b: VERO cells in the absence of the composition according to the invention: Ig TCID50=7.13
Condition b−Condition a=0<1 lg

The sensitivity is therefore valid.
Checking the effectiveness of disinfectant shutdown at T=0
Condition a: VERO cells in the presence of the composition according to the invention: Ig TCID50=6.75
Condition b: VERO cells in the absence of the composition according to the invention: Ig TCID50=6.88
Difference <0.5 lg, therefore valid control.

### Test Viral Control:

Before Sephadex: Ig TCID50=6
After Sephadex: Ig TCID50=5.75
Difference=0.25<0.5 lg so the control is valid

TABLE 4
Coronavirus 229 E - 60 minutes ± 10 seconds

| | | | | | | | Control | |
|---|---|---|---|---|---|---|---|---|
| | In the presence of compositions 5, 6 or 7 | | | | | | Degree of | % of |
| −log | Degree of cytopathic effect | | | % of positive wells | | | cythopathic | positive |
| dilution | 7 | 6 | 5 | 7 | 6 | 5 | effect | wells |
| 1 | 11111111 | 11111111 | 11111111 | 100% | 100% | 100% | 11111111 | 100% |
| 2 | 11111111 | 11111111 | 11111111 | 100% | 100% | 100% | 11111111 | 100% |
| 3 | 11000000 | 11111111 | 11111111 | 25% | 100% | 100% | 11111111 | 100% |
| 4 | 00000000 | 11000000 | 11111111 | 0% | 25% | 100% | 11111111 | 100% |
| 5 | 00000000 | 00000000 | 11111000 | 0% | 0% | 62.5% | 11111110 | 87.5% |
| 6 | 00000000 | 00000000 | 00000000 | 0% | 0% | 0% | 11100000 | 37; 5% |
| 7 | 00000000 | 00000000 | 00000000 | 0% | 0% | 0% | 000000000 | 0% |
| 8 | 00000000 | 00000000 | 00000000 | 0% | 0% | 0% | 000000000 | 0% |
| Lg | | | | 2.75 | 3.75 | 5.12 | | 5.75 |
| TCID50 | | | | | | | | |

* 1 to 4: Presence of virus, with cytopathic effect observed in 8 cell wells 0: Virus-free, with no cytopathic effect

### CONCLUSION

TABLE 5

| Contact time | 60 min ± 10s | | | | | | |
|---|---|---|---|---|---|---|---|
| Composition | 1 | 2 | 3 | 4 | 5 | 6 | 7 |
| Lg reduction | 0.63 | 0.88 | 1.5 | 1.25 | 0.63 | 2 | 3 |
| % lg reduction | 76.56% | 86.82% | 96.84% | 94.38% | 76.56% | 99% | 99.9% |

The results of the reduction in the viral load of Coronavirus 229 E after a contact time of 60 min with the product chitosan alone (composition 1 or 2), the combination of chitosan+hops (composition 3 or 4) and xanthohumolone alone (compositions 5 to 7) show a lower cytopathic effect on Coronavirus 229 E than that of the chitosan/xanthohumolone combination according to the invention.

1. A composition comprising a mixture of at least chitosan and Xanthohumolone.

2. The composition according to claim 1, characterized in that it further comprises a biocompatible solvent.

3. The composition according to claim 2, characterized in that the biocompatible solvent is chosen from acetic acid, lactic acid, glycolic acid, glutamic acid, glycerol or mixtures thereof.

4. The composition according to claim 1, characterized in that the xanthohumolone/chitosan ratio is between 0.2 and 0.7.

5. The composition according to claim 1, characterized in that the chitosan has a viscosity between 70 and 150 cP.

6. The composition according to claim 1, characterized in that the chitosan has a molecular weight between 150 and 500 KDa.

7. The composition according to claim 1, characterized in that it is in the form of a gel, solution, spray, or cream.

8. The composition according to claim 1, for use thereof as a medication.

9. The composition according to claim 1, for use thereof as an antiviral and/or antibacterial composition.

10. The composition according to claim 1, for use thereof the treatment and/or prevention of viral and/or bacterial illnesses.

11. The composition according to claim 1, for use thereof in the treatment and/or prevention of COVID-19 and all its variants.

12. A use of a composition according to claim 1 in the treatment of medical devices.

13. A kit comprising the composition according to claim 1 and a vaccine.

14. A medical device comprising the composition of claim 1.
